(19) United States
(12) Patent Application Publication
(10) Pub. No.: US 2006/0084700 A1
(43) Pub. Date: Apr. 20, 2006

(54) Use of a beta-3 agonist for the treatment of disorders of the prostate and of the lower urogenital tract

(75) Inventor: Martin Christian Michel, Amsterdam (NL)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/252,838

(22) Filed: Oct. 18, 2005

Related U.S. Application Data
(60) Provisional application No. 60/624,590, filed on Nov. 3, 2004.

(30) Foreign Application Priority Data
Oct. 18, 2004 (DE) 10 2004 050 952

(51) Int. Cl. A61K 31/24; A61K 31/195

(52) U.S. Cl. 514/534; 514/567

(57) ABSTRACT

This invention describes the use of beta-3-adrenoceptor agonists for the treatment of disorders associated with the prostate. These include disorders like those occurring in a prostatitis, where attributable to inflammatory processes or chronic irritation, or disorders like those associated with benign changes of the prostate. The invention is particularly suitable for the treatment of benign prostatic hyperplasia (BPH).

### BACKGROUND OF THE INVENTION

1. Field of the Invention

This invention relates to the use of beta-3 adrenoceptor agonists for the treatment of disorders associated with pathological changes or irritation of the prostate. These include disorders like those associated with benign changes in the prostate, especially benign prostatic hyperplasia (BPH) or disorders like those occurring in association with a prostatitis, whether attributable to inflammatory processes or to chronic irritation.

2. Description of the Prior Art

Benign prostatic hyperplasia (BPH) is a disease of unknown etiology which occurs in more than 50% of men over 50 years of age and leads to an enlargement of the prostate. The BPH symptom complex may be associated with benign prostatic enlargement (BPE), obstructive voiding impairments (bladder outlet obstruction or BOO for short) and irritative symptoms of the lower urogenital tract. Besides the term BPH, also to be found is the term benign prostatic syndrome—BPS, a generic term for the pathophysiologically very variable relation between the symptoms of irritative disorders in the lower urinal tract, prostatic enlargement (BPE) and obstruction (BOO or BPO). The symptoms suffered by quite a large proportion of BPH patients are mainly irritative disorders in the lower urogenital tract (LUTS) and only slight obstruction.

A suggested cause of BPH is, inter alia, an increase in the number of cells of the prostate, the size of which remains unchanged, however. One of the results of enlargement of the prostate may be constriction of the urethra in this region, and complete emptying of the bladder may be impeded. In addition, impairments of bladder function may be associated with the disease and may enhance the irritative symptoms. It is also possible for overflow incontinence to develop, or total retention of urine. It is also possible as a consequence thereof for neighboring organs such as the kidney to be affected (e.g., hydronephrosis, progressive renal failure). The risk of developing an acute or chronic urinary tract infection is often increased in the presence of a benign prostatic hyperplasia.

Functional symptoms of benign prostatic hyperplasia (BPH) are treated by using alpha-adrenoceptor antagonists and 5-alpha-reductase inhibitors. Representatives of the class of alpha-adrenoceptor antagonists are able to bind selectively and competitively to the post-synaptic alpha-1 receptors. The smooth muscles of the prostate and of the urethra are relaxed thereby, and the tone of the smooth muscles of the prostate and urethra is reduced. As a result of this, the urinary flow rate is increased. 5-Alpha-reductase inhibitors inhibit the enzyme 5-alpha-reductase. This enzyme converts the endogenous testosterone into dihydrotestosterone, which directly stimulates the growth of prostatic tissue.

Similar symptoms like those of BPH may also develop within the framework of other pathological prostatic processes such as, for example, with a prostatitis. The term prostatitis itself encompasses a heterogeneous pathological state with multiple causes which often are or remain unrecognized. The current National Institutes of Health (NIH) classification differentiates four categories of prostatitis: acute prostatitis (NIH I), chronic bacterial prostatitis (NIH II), chronic nonbacterial prostatitis (NIH III), and asymptomatic prostatitis (NIH IV). Chronic nonbacterial prostatitis (NIH III) is also referred to as chronic pelvic pain syndrome and is in turn divided into a chronic nonbacterial inflammatory form (NIH IIIa) and a noninflammatory pain syndrome (NIH IIIb). Whereas the diagnosis of acute prostatitis (NIH I) can usually be made unambiguously, the differential diagnosis of the chronic forms is difficult. Bacterial prostatitis (NIH I and II) may be initiated by urogenous or hematogenous infections or else by spread of an inflammation from neighboring organs. Acute bacterial prostatitis may subsequently develop into an inflammatory chronic prostatitis, which may remain bacterial or become nonbacterial. However, the cause of a nonbacterial prostatitis can in many cases not be identified unambiguously, but various neurogenic and muscular initiating factors are suggested. The neurogenic causes include for example neuropathies and inflammations of nerves, especially in the region of the true pelvis, but also in the region of the false pelvis, the adjacent areas of intestine or in the region of the anus. The muscular initiating factors include involuntary and frequent contraction of the muscles of the pelvic floor, of the lumbar muscles and of other muscles located in the vicinity of the prostate. This persistent contracting of muscles with few or no phases of muscular relaxation may occur as involuntary response to phases of stress, aggressiveness, frustration and the like. Tensing of the muscles of the pelvic floor may also be the consequence of prolonged phases of sitting and other one-sided postures such as bicycle riding etc. This form of prostatitis is also referred to as non-inflammatory chronic pelvic pain syndrome, pelvic myoneuropathy, prostatodynia, or prostatopathy.

The symptoms of prostatitis are similar to the symptoms of BPH or LUTS. They include dysuria, pollakiuria, pain during defecation, retention of urine, burning during urination, pain and discomfort in the vicinity of the prostate, pain on ejaculation, and the like.

Selective beta-3-adrenoceptor agonists are being discussed in relation to their suitability for various areas of indication. These include, inter alia, obesity, diabetes, and incontinence of urine. The use of selective beta-3-adrenoceptor agonists in the therapy of incontinence of urine has been known since 1995 (EP 0 958 835).

### SUMMARY OF THE INVENTION

The present invention is based on the object of treating disorders in the lower urogenital tract, especially the prostate, which are attributable to acute or, preferably, chronic inflammations or irritation of the prostate or to benign prostatic enlargement (BPH).

One object of the invention relates to the treatment of BPH in all its symptomatic manifestations.

A further object relates to the treatment of acute or, preferably, chronic prostatitis.

A further object relates to the treatment of the chronic pelvic pain syndrome, of pelvic myoneuropathy, of prostatodynia, or of prostatopathy.

A further object relates to the treatment of obstructive bladder emptying impairments (BOO) in men.

A further object relates to the treatment of the symptom complex of LUTS (lower urinary tract symptoms) in men.

In this connection, the use of beta-3-adrenoceptor agonists and pharmaceutical compositions which comprise compounds from this class of active ingredients is presented according to the invention.

### DESCRIPTION OF THE INVENTION

The present invention provides a novel pharmaceutical composition which comprises at least one beta-3-adrenoceptor agonist in a pharmaceutically effective amount as active ingredient.

a) Active Components

The preferred active components are specified below. Where any pharmaceutically active compound is disclosed or claimed, it is expressly intended to include all active metabolites generated in vivo, and it is expressly intended to include all possible stereoisomers or tautomers. Likewise included are pharmaceutically acceptable salts thereof. Examples of acids which may be mentioned for salt formation for the basic compounds are: acetic, benzenesulphonic (besylate), benzoic, p-bromophenylsulphonic, camphorsulphonic, carbonic, citric, ethanesulphonic, fumaric, gluconic, glutamic, hydrobromic, hydrochloric, hydroiodic, isethionic, lactic, maleic, malic, mandelic, methanesulphonic (mesylate), mucic, nitric, oxalic, pamoic, pantothenic, phosphoric, succinic, sulphuric, tartaric, p-toluenesulphonic acids, and the like.

Where necessary for completeness, the synthesis of the compounds for which the prior art is stated, and the dosages thereof are expressly included by reference to the prior art cited at the appropriate point.

The beta-3-adrenoceptor agonists used according to the invention are preferably phenoxyacetic acid derivatives. These are preferably selected from the following group according to formula I: 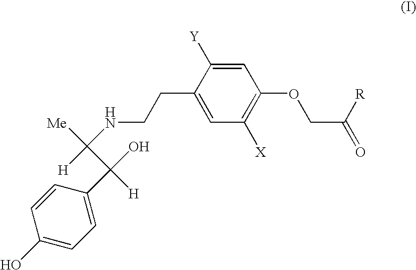
with
1) X=Br, Y=H, R=OH
2-[2-bromo-4-[2-[[(1S,2R)-2-hydroxy-2-(4-hydroxyphenyl)-1-methylethyl]amino]ethyl]phenoxy]acetic acid,
2) X=Cl, Y=H, R=OH
2-[2-chloro-4-[2-[[(1S,2R)-2-hydroxy-2-(4-hydroxyphenyl)-1-methylethyl]amino]ethyl]-phenoxy]acetic acid,
3) X=Y=Cl, R=OH
2-[2,5-dichloro-4-[2-[[(1S,2R)-2-hydroxy-2-(4-hydroxyphenyl)-1-methylethyl]amino]ethyl]phenoxy]acetic acid,
4) X=Y=H, R=OH
2-[4-[2-[[(1S,2R)-2-hydroxy-2-(4-hydroxyphenyl)-1-methylethyl]amino]ethyl]-2,5-dimethylphenoxy]acetic acid,
5) X=OH; Y=H; R=OH
2-[2-hydroxy-4-[2-[[(1S,2R)-2-hydroxy-2-(4-hydroxyphenyl)-1-methylethyl]amino]ethyl]phenoxy]acetic acid,
6) X=Cl; Y=H, R=OEt ethyl 2-[2-chloro-4-[2-[[(1S,2R)-2-hydroxy-2-(4-hydroxyphenyl)-1-methylethyl]amino]ethyl]phenoxy]acetate,
7) X=Cl; Y=Cl, R=OEt ethyl 2-[2,5-dichloro-4-[2-[[(1S,2R)-2-hydroxy-2-(4-hydroxyphenyl)-1-methylethyl]amino]ethyl]phenoxy]acetate,
8) X=Me; Y=Me, R=OEt Ethyl (−)-2-[4-(2-{[(1S,2R)-2-hydroxy-2-(4-hydroxyphenyl)-1-methylethyl]amino}ethyl)-2,5-dimethylphenyloxy]acetate, and the corresponding hydrochloride.
9) X=Me; Y=Me, R=OH
(−)-2-[4-(2-{[(1S,2R)-2-hydroxy-2-(4-hydroxyphenyl)-1-methylethyl]amino}ethyl)-2,5-dimethylphenyloxy]acetic acid,

These mentioned compounds are disclosed in WO 00/02846 or WO 2003024916.

Additionally of interest are the following compounds: 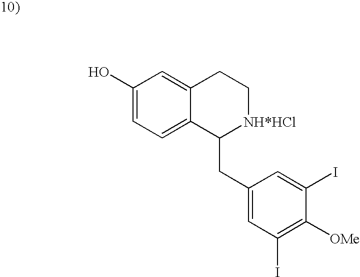
or the free base thereof.

Name: 1-(4-methoxy-3,5-diiodophenyl)-methyl-1,2,3,4-tetrahydroisoquinolin-6-ol or the hydrochloride, J. Med. Chem. 44 (2001) 1456.

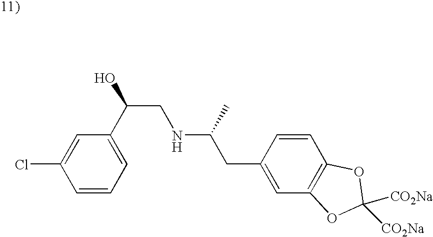

Name: disodium-([R,R]-5-2-[[2-(3-chlorophenyl)-2-hydroxyethyl]amino]propyl)-1,3-benzodioxol-2,2-dicarboxylate or the hydrochloride, J. Med. Chem. 44 (2001) 1456; Journal of Urology 165 (2001) 240.

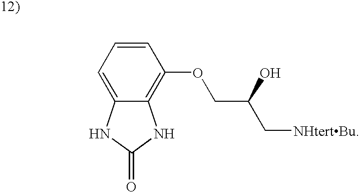

Name: 4-((3-N-tert-butylamino)-2-hydroxypropyloxy)-1,3-dihydrobenzoimidazol-2-one or the hydrochloride, Journal of Urology 165 (2001) 240, J. Med. Chem. 44 (2001) 1456.

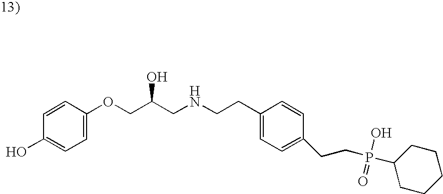

Name: cyclohexyl[[4-[2-[[(2S)-2-hydroxy-3-(4-hydroxylphenoxy)propyl]amino]ethyl]-phenoxy]methyl]phosphinic acid, J. Med. Chem. 44 (2001) 1456.

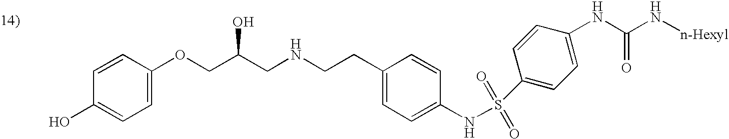

Name: (S)-4-[[(hexylamino)carbonyl]amino]-N-[4-[2-[[2-hydroxy-3-(4-hydroxyphenoxy)-propyl]amino]ethyl]phenyl]benzenesulphonamide, J. Med. Chem. 44 (2001) 1456.

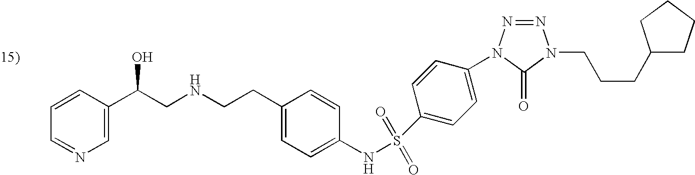

Name: (R)-4-[4-(3-cyclopentylpropyl)-4,5-dihydro-5-oxo-1H-tetrazol-1-yl]-N-[4-[2-[[2-hydroxy-2-(3-pyridinyl)ethyl]amino]ethyl]phenyl]benzenesulphonamide or the hydrochloride, J. Med. Chem. 44 (2001) 1456.

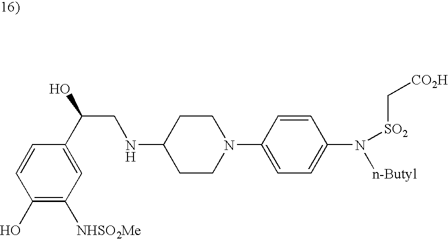

Name: 4-(N-(2-(4-hydroxy-3-methylsulphonamido-phenyl)-2-hydroxy-ethyl)-amino)-piperidin-1-yl-phenyl-4-yl-(n-butylamino)-sulphonyl-acetic acid, J. Med. Chem. 44 (2001) 1456, Bioorg. Med. Chem. Lett. 9 (2001) 2045. 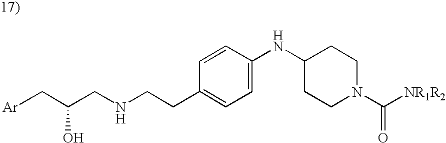
or the hydrochloride
with
Ar=4-HO-Ph-O, R1=octyl, R2=H, Name: 4-(4-(2-(3-(4-hydroxyphenoxy)-2-hydroxypropyl)aminoethyl)anilino)piperidinylcarbonyl-N-octylamide;
Ar=4-HO-3-methylsulphonylamidophenyl-O, R1=2,5-difluorobenzyl, R2=H;
Name: 4-(4-(2-(3-(4-hydroxy-3-methylsulphonylamidophenoxy)-2-hydroxypropyl)-aminoethyl)anilino)piperidinylcarbonyl-N-(2,5-difluorobenzyl)amide;
Ar=4-HO-3-methylsulphonylamidophenyl, R1=2,5-difluorobenzyl, R2=H, Name: 4-(4-(2-(3-(4-hydroxy-3-methylsulphonylamidophenyl)-2-hydroxypropyl)amino-ethyl)anilino)piperidinylcarbonyl-N-(2,5-difluorobenzyl)amide; (Bioorg. Med. Chem. Lett. 11 (2000) 3123). 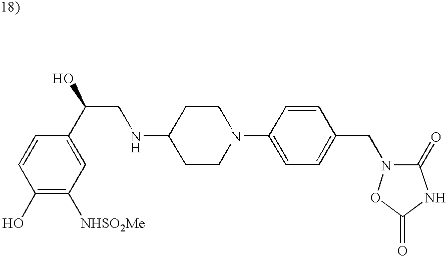
or the hydrochloride

Name: 2-(4-N-(4-(2-(4-hydroxy-3-methylsulphonylamidophenyl)-2-hydroxyethyl)amino)-piperidinyl)benzyl)-[1,2,4]oxadiazolidine-3,5-dione, Bioorg. Med. Chem. Lett. 11 (2001) 981. 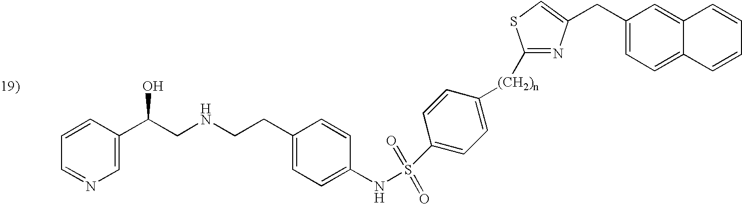
or the hydrochloride.

n may be 0 or 1;
Name: (4-(4-(2-pyridinyl-2-hydroxyethylaminoethyl)anilino)sulphonyl)-2-benzyl-4-naphth-2-ylmethylthiazole (n=1) and (4-(4-(2-pyridinyl-2-hydroxyethylaminoethyl)anilino)-sulphonyl)-2-phenyl-4-naphth-2-ylmethylthiazole (n=0), Bioor. Med. Chem. Lett. 10 (2000) 1971. 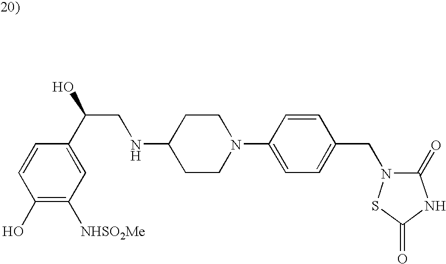
or the hydrochloride.

Name: 2-(4-N-(4-(2-(4-hydroxy-3-methylsulphonylamidophenyl)-2-hydroxyethyl)amino)-piperidinyl)benzyl)-[1,2,4]thiadiazolidine-3,5-dione, Bioorg. Med. Chem. Lett. 11 (2001) 757. 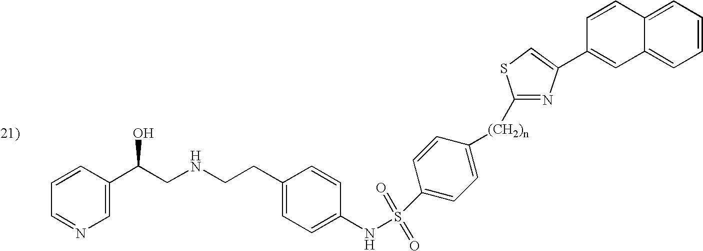
or the hydrochloride.

n may be 0 or 1.

Name: (4-(4-(2-pyridinyl-2-hydroxyethylaminoethyl)anilino)sulphonyl)-2-benzyl-2-naphthyl-thiazole (n=1) and (4-(4-(2-pyridinyl-2-hydroxyethylaminoethyl)anilino)sulphonyl)-2-phenyl-2-naphthylthiazole (n=0), Bioor. Med. Chem. Lett. 10 (2000) 1971. 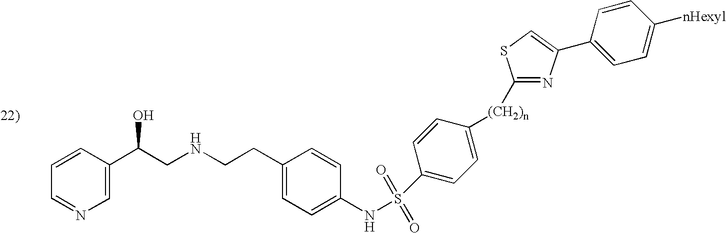
or the hydrochloride.

Name: (4-(4-(2-pyridinyl-2-hydroxyethylaminoethyl)anilino)sulphonyl)-2-benzyl-(4-n-hexyl-phenyl)thiazole (n=1) and (4-(4-(2-pyridinyl-2-hydroxyethylaminoethyl)anilino)sulphonyl)-2-phenyl-(4-n-hexylphenyl)thiazole (n 0), Bioor. Med. Chem. Lett. 10 (2000) 1971. 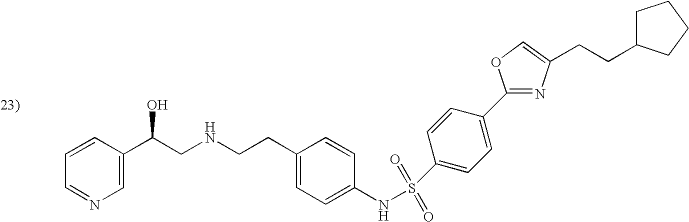
or the hydrochloride.

Name: 2-(4-(4-(2-pyridinyl-2-hydroxyethylaminoethyl)anilino)sulphonyl)phenyl-4-(cyclopentylethyl)oxazole, Bioorg. Med. Chem. Lett. 10 (2000)1531. 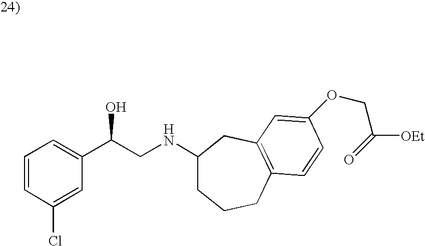
or the hydrochloride.

Name: Ethyl [R—(R*,S*)]-[[8-[[2-(3-chlorophenyl)-2-hydroxyethyl]amino]-6,7,8,9-tetrahydro-5H-benzocyclohepten-2-yl]oxy]acetate, hydrochloride, 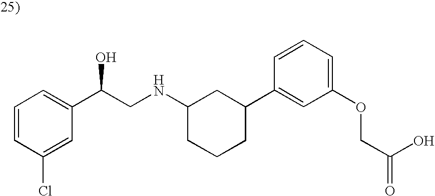
or the hydrochloride.

Name: [1S-[1α,3β(S*)]]-3-[3-[[2-(3-chlorophenyl)-2-hydroxyethyl]amino]cyclohexyl]phenoxy]acetic acid, monosodium salt, 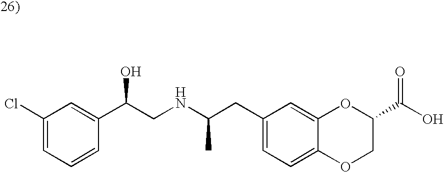
or the hydrochloride.

Name: 6-[(2R)-2-[[(2R)-2-(3-chlorophenyl)-2-hydroxyethyl]amino]propyl]-2,3-dihydro-(1,4-benzodioxin-2-carboxylic acid).

27)

2-(3-{[2-(3-chlorophenyl)-2R-hydroxylethylamino]ethylamino}phenyl)thiophene-3-carboxylic acid or the hydrochloride. 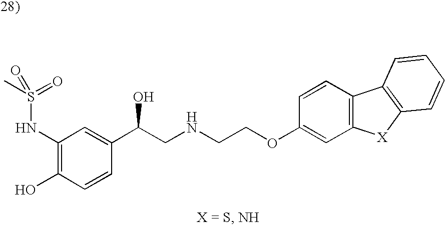
or the hydrochloride.

Name: 3-(1-(4-hydroxy-3-methylsulphonylamidophenyl)-1-hydroxyethylamino)ethoxy)-dibenzothiophene and 2-(1-(4-hydroxy-3-methylsulphonylamidophenyl)-1-hydroxyethyl-amino)ethoxy)-9H-carbazole. 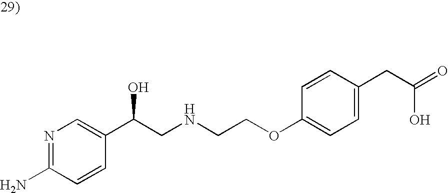
or the hydrochloride.

Name: [4-[2-[[2-(6-aminopyridin-3-yl)-2(R)-hydroxyethyl]amino]ethoxy]phenyl]acetic acid. 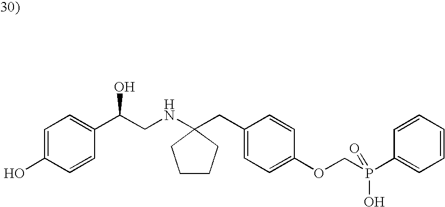
or the hydrochloride.

Name: [[4-[[1-[[(2S)-2-hydroxy-3-(4-hydroxyphenyl)-propyl]amino]cyclopentyl]-methyl]phenoxy]methyl]phenyl-phosphinic acid. 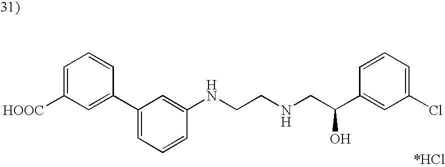
or the free base.

Name: [1,1′-biphenyl]-3-carboxylic acid, 3′-[[2-[[(2R)-2-(3-chlorophenyl)-2-hydroxyethyl]amino]ethyl]amino] hydrochloride or solabegron. 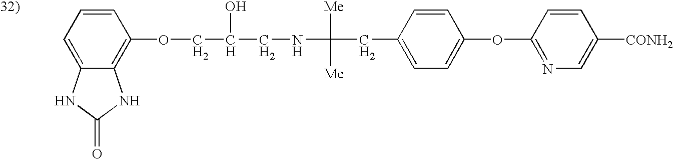
or the hydrochloride.

Name: 6-[4-[2-[[3-[(2,3-dihydro-2-oxo-1H-benzimidazol-4-yl)oxy]-2-hydroxypropyl]amino]-2-methyl propyl]phenoxy]-3-pyridinecarboxamide. 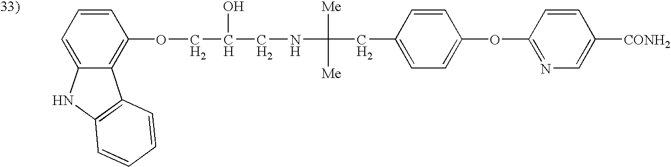
or the hydrochloride.

Name: (S)-6-[4-[2-[[3-(9H-carbazol-4-yloxy)-2-hydroxypropyl]amino]-2-methylpropyl]phenoxy]-3-pyridinecarboxamide.

b) Dosage

In order to determine the optimal dose of active ingredient it is necessary to take account of various limiting conditions such as, for example, age and body weight of the patient, nature and stage of the disease, and the potency of the compound. This is regarded as being within the capability of the skilled person; the existing literature about the components may be consulted in order to determine the optimal dosage. The stated dosages relate to the dosage after completion of the stabilization phase.

The dosages stated below expressly include all numerical values, whole or fractional, within the stated range. The data relate to adult people. Pediatric dosages may be smaller.

More than once daily or twice daily administrations (e.g., 3, 4, 5, or 6 administrations per day) are likewise expressly taken into consideration herein.

An amount smaller than that stated may also suffice in some cases, whereas a larger total amount may be necessary in other cases.

The total daily dose may, depending on the therapy regimen, be taken all at once or within a plurality of portions. The therapy regimen may also specify intervals longer than one day between the intakes.

The average daily dose of the beta-3-agonist for an adult man may be from about 1 mg to 1000 mg, preferably 10 mg to about 750 mg per day, preferably 20 to 500 mg, more preferably 20 to 200 mg. This amount is preferably administered as a dose once or twice a day.

c) Administration Forms

The compositions of the present invention can expediently be administered in a pharmaceutical composition which comprises the active component in combination with a suitable carrier. Such pharmaceutical compositions can be produced by processes, and comprise carriers, which are well known in the art. Generally acknowledged specialist works are available to the skilled person in this regard.

The compositions of the present invention can be administered parenterally (e.g., by intravenous, intraperitoneal, subcutaneous, or intramuscular injections), topically, orally, intranasally, transdermally, rectally, by pulmonary inhalation, or by nasal inhalation, with particular preference for oral administration. Among the oral administration forms, preference may be given to formulations resistant to gastric juice. In this case, capsules resistant to gastric juice or tablets resistant to gastric juice are preferred, it being possible in both cases to achieve this by, for example, a coating resistant to gastric juice. The skilled person will find instructions for formulations resistant to gastric juice in the state of the art.

Various formulation options are given below. The skilled person can select therefrom a suitable formulation.

For oral therapeutic administration, the composition according to the invention can be combined with one or more carriers and be used in the form of tablets which can be taken, buccal tablets, sublingual tablets, sugar-coated tablet, oral powders, dusting powders, pastilles, coated tablets, granules, capsules, elixirs, suspensions, solutions, syrups, wafers, chewing gums, food products, and the like.

A powder may be produced for example by bringing the particles of the active substance to a suitable size by grinding.

Diluted powders can be produced by finely grinding the substance in powder form with a nontoxic carrier material such as, for example, lactose, and applying as powder. Other carrier materials suitable in this regard are other carbohydrates, such as starch or mannitol. These powders may where appropriate comprise flavorings, preservatives, dispersing agents, colors, and other pharmacological excipients.

Capsules may be produced starting from a powder of the abovementioned type or other powders, which are introduced into a capsule, preferably a gelatin capsule, and the capsule is then closed.

It is also possible for lubricants known in the state of the art to be introduced into the capsule or to be used for sealing the two parts of the capsule. The efficacy of a capsule on oral intake can be enhanced by adding disintegrating or solubilizing substances such as, for example, carboxymethylcellulose, carboxymethylcellulose calcium, low-substituted hydroxypropylcellulose, calcium carbonate, sodium carbonate, and other substances. The active ingredient may be present in the capsule not only as solid but also as suspension, for example in vegetable oil, polyethylene glycol, glycerol with the aid of surface-active substances etc.

Tablets may be produced by compressing the mixture in powder form, and subsequently further processing for example to granules. The tablets may comprise various excipients such as, for example, starches, lactose, sucrose, glucose, sodium chloride, urea for tablets for solution and injection, amylose, various types of cellulose as described above and others.

Humectants which can be used are, for example, glycerol or starch.

Disintegrants which can be used are for example starch, alginic acid, calcium alginate, pectic acid, powdered agar-agar, formaldehyde gelatin, calcium carbonate, sodium bicarbonate, magnesium peroxide, and amylose.

Suitable antidisintegrants or solution retarders are, for example, sucrose, stearin, solid paraffin (preferably with a melting range of 50-52° C.), cocoa fat, and hydrogenated fats.

Further disintegrants may be: maize starch, potato starch, alginic acid, and the like.

Suitable absorption promoters are, inter alia, quaternary ammonium compounds, sodium lauryl sulphate, saponins.

It is possible to use as binder distributors for example ethers, and as hydrophilizing agents or as disintegration promoters cetyl alcohol, glycerol monostearate, starch, maize starch, lactose, wetting agents (e.g., Aerosol OT, Pluronics, Tweens), gum tragacanth, gum arabic, gelatine, and others.

It is possible to employ as sweeteners sucrose, fructose, lactose, or aspartame, or as flavorings peppermint, oil of wintergreen, cherry flavor, and many others.

The above listing is merely by way of example, and a skilled person would be able to consider other excipients from the state of the art.

Tablets can be produced for example by direct compression.

Tablets and similar solid forms which can be administered orally may be provided with coatings. For example, tablets, pills, or capsules can be coated with gelatin, wax, shellac or sugar and the like. As already mentioned, formulations resistant to gastric juice are preferred for the oral dosage forms. Hence, coatings resistant to gastric juice are preferred for tablets or capsules. In the case of a syrup or elixir, sucrose or fructose may be present as sweetener, methyl paraben and propyl paraben as preservative, a color, and a flavoring, such as cherry or orange flavor.

It is also possible to produce other formulations which can be administered orally, such as solutions, syrup, elixir, etc. The compound may where appropriate be microencapsulated.

A parenteral administration can be achieved by the compound being dissolved in a liquid and injected subcutaneously, intramuscularly, or intravenously. Examples of suitable solvents are water or oily media.

Suppositories can be produced by formulating the compound with low-melting and water-soluble or water-insoluble materials such as polyethylene glycol, cocoa butter, higher esters (for example moerysthyl, palmitate), or mixtures thereof.

Every material which is used in the production of every unit dose form should, of course, be pharmaceutically acceptable and essentially non-toxic in the amounts used. In addition, the active components can be incorporated into products with delayed release and devices which, without being restricted thereto, include those which are based on osmotic pressures, in order to achieve a desired release profile. Once-a-day formulations for each of the active components are specifically included.

Compositions and products of these types should comprise at least 0.001% of active compound. The percentage of the compositions and products can, of course, be varied and may expediently amount to between about 0.1 to about 100% of the weight of a given unit dose form. The amount of active compound in therapeutically utilizable compositions of these types is such that an effective dosage amount is obtained.

d) Indications

Each of the compounds listed as beta-3-adrenoceptor agonists can be employed according to the invention for the treatment or prophylaxis, inter alia, of any of the pathological states mentioned below, as single pathological state and in combination with any other of the pathological states mentioned, provided that they comprise irritative symptoms or diseases of the lower urinary tract of men, especially of the prostate or corresponding accompanying symptoms: benign prostatic hyperplasia, prostatitis, especially chronic nonbacterial prostatitis, of neurogenic, muscular or bacterial origin, chronic pelvic pain syndrome, pelvic myoneuropathy, prostatodynia, LUTS (lower urinary tract symptoms), obstructive bladder emptying impairments (BOO) and/or prostatopathy. The use according to the invention is aimed not only at causative treatment of the pathological change of the prostate or of the pelvic muscles which is associated with the indications mentioned, but also at the treatment of the accompanying symptoms, especially the pain associated where appropriate therewith or the problems of discharging urine. These include dysuria, pollakiuria, retention of urine, suppressible, imperative urge to urinate associated with or without urge incontinence, increased frequency of urination, nocturnal urination (dysuria and nycturia), incomplete bladder emptying, burning during urination, pain and discomfort in the vicinity of the prostate or of the lower urinary tract including the penis, pain on erection or ejaculation, pain during defecation, erectile dysfunctions.

By causative treatment is meant that the pathological state progresses or is improved in this sense on administration of the medication according to the invention. By symptomatic treatment is meant that the disorders associated with the accompanying symptoms are perceived less or the disorders are alleviated.

The pathological states encompassed in this connection according to the invention are both those caused by an organic dysfunction or disease and those whose cause is direct from the bacterial inflammation, mechanical overstrain or from diseases or impairments of the central and/or peripheral nervous system.

Hence, a further embodiment of the present invention comprises the use of the composition according to the invention for producing a medicament for the treatment or prevention of any of the indications mentioned in the preceding paragraph.

The above diseases or impairments are treated by giving a therapeutically effective amount of the composition according to the invention to a mammal. In most cases, this is a human, but the treatment of food animals (e.g., cattle) and domestic animals (e.g., dogs, cats, and horses) is expressly covered herein. The dosages to be used for the veterinary uses may be different from the dosages indicated herein.

It is expected that the novel composition will ensure, with a minimal degree of harmful side effects, rapid alleviation for those suffering from the above diseases and impairments.

e) Combinations

According to the present use according to the invention, the beta-3-adrenoceptor agonist can also be combined with other active ingredients. Some combination partners are mentioned below. The active ingredients may where appropriate be used in the form of the neutral compound or in the form of salts. Some possibilities are mentioned by way of example, but not exclusively.

Preferred examples of combination partners mentioned are:

Alpha 1-adrenoceptor antagonist, such as tamsulosin, tamsulosin hydrochloride, alfuzosin, bunazosin, doxazosin, indoramin, naftopidil, prazosin, terazosin, urapidil, silodosin, moxisylyte, metazosin, fiduxosin, upidosin, SNAP-5089 (5-(N-(3-(4,4-diphenylpiperidin-1-yl)propyl)carbamoyl)-2,6-dimethyl-4(R)-(4-nitrophenyl)-1,4-dihydropyridine-3-carboxylic acid methyl ester), AIO-8507L, SL-890591 ((2-(3-(4-(5-chloro-2-methoxyphenyl)piperazine-1-yl)propylamino)pyrimidine-4-carboxamide fumarate), and RS-100329 (5-methyl-3-(3-(4-(2-(2,2,2-trifluoroethoxy)phenyl)piperazine-1-yl)propyl)pyrimidine-2,4(1H,3H)-dione hydrochloride);

Antimuscarinics, such as (S)-N-{3-[4-(2-(2,3-dihydrobenzofuran-5-yl)-1-methylethyl)-ethylamino]methylpiperidin-1-yl]-3-oxopropyl}methanesulphonamide, [1,1′-biphenyl]-2-ylcarbamic acid 1-azabicyclo[2.2.2]oct-4-yl-ester monohydrochloride, 2-methyl-alpha,alpha-diphenyl-1H-imidazole, AH-9700, benzohydrylcarbamic acid N-(4-methylaminobenzyl)piperidin-4-yl ester, bethanchol chloride, darifenacin, darifenacin chloride, dicyclomine hydrochloride, emepronium chloride, fesoterodin, FK-584, hyoscyamine sulphate, imipramine hydrochloride, oxybutynin chloride, S-oxybutynin chloride, ipratropium, J-104135, N-[2-(2,3-dihydrobenzofuran-5-yl)-1-methylethyl]-N-ethyl-(1-methanesulphonylpiperidin-4-ylmethyl)-amine, N-ethyl-N-[2-(4-methoxyphenyl)-1-methylethyl)-[1-(dimethylaminocarbonyl)-piperidin-4-ylmethyl]amine, oxybutynin, propantheline bromide, propiverine, propiverine chloride, revatropate chloride, solifenacin, temiverine, temiverine chloride, terodiline chloride, tolteridine tartrate, tolterodine, trospium, trospium chloride, and vamicamide chloride.

1. A method for the prophylaxis or treatment of an irritative symptom or disease of the lower urinary tract in men comprising administering a medicament comprising a beta-3-adrenoceptor agonist.

2. The method of claim 1, wherein the irritative symptom or disease of the lower urinary tract is an irritative symptom or disease of the prostate.

3. The method of claim 1, wherein the irritative symptom or disease of the lower urinary tract is BPH.

4. The method of claim 1, wherein the irritative symptom or disease of the lower urinary tract is prostatitis

5. The method of claim 4, wherein the prostatitis is chronic nonbacterial prostatitis.

6. The method of claim 1, wherein the irritative symptom or disease of the lower urinary tract is LUTS.

7. The method of claim 1, wherein the irritative symptom or disease of the lower urinary tract is chronic pelvic pain syndrome, pelvic myoneuropathy, prostatodynia, obstructive bladder emptying impairments (BOO), or prostatopathy.

8. The method of claim 1, wherein the beta-3-adrenoceptor agonist is a compound according to formula I 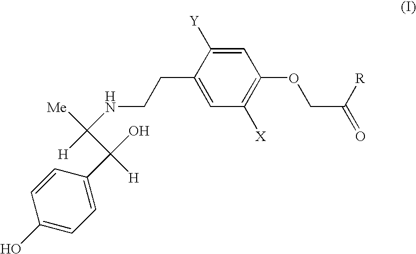
or a pharmaceutically acceptable salt thereof,
wherein X is H, Cl, Br, OH, or methyl; Y is H, Cl, Br, OH, or methyl; and R is OH, methyl, ethyl, or OEt.

9. The method of claim 8, wherein X is Br, Y is H, and R is OH.

10. The method of claim 8, wherein X is Cl, Y is H, and R is OH.

11. The method of claim 8, wherein X and Y are Cl, and R is OH.

12. The method of claim 8, wherein X and Y are H, and R is OH.

13. The method of claim 8, wherein X is OH; Y is H; and R is OH.

14. The method of claim 8, wherein X is Cl, Y is H, and R is OEt, or the corresponding hydrochloride.

15. The method of claim 8, wherein X and Y are Cl, and R is OEt, or the corresponding hydrochloride.

16. The method of claim 8, wherein X and Y are Me, and R is OEt, or the corresponding hydrochloride.

17. The method of claim 8, wherein X and Y are Me, and R is OH.

18. The method of claim 1, wherein the beta-3-adrenoceptor agonist is used in an amount of from about 10 mg to about 750 mg.

19. The method of claim 1, wherein the medicament is adapted for rectal, topical, oral, sublingual, intranasal, transdermal, or parenteral administration.
